(19) United States
(12) Patent Application Publication
(10) Pub. No.: US 2026/0286843 A1
(43) Pub. Date: Sep. 24, 2026

(54) REAL TIME DOWNHOLE API DETERMINATION

(71) Applicant: Schlumberger Technology Corporation, Sugar Land, TX (US)

(72) Inventors: Talha Zubair, Dhahran (SA); Ahmed Moge Ali, Dhahran (SA); Mikhail Charupa, Dhahran (SA); Endurance O Ighodalo, Dhahran (SA); Mustapha Berkane, Dhahran (SA); Ashers Partouche, Sugar Land, TX (US); Francois-Xavier Dubost, Paris (FR); Adriaan Gerard Gisolf, The Hague (NL); Deopaul Dindial, Dhahran (SA)

(21) Appl. No.: 19/543,631

(22) Filed: Feb. 18, 2026

Related U.S. Application Data
(60) Provisional application No. 63/759,669, filed on Feb. 18, 2025.

(51) Int. Cl. E21B 49/08; G01N 5/00; G01N 9/36; G01N 33/28

(52) U.S. Cl. CPC E21B 49/082; E21B 49/0875; E21B 49/088; G01N 5/00; G01N 9/36; G01N 33/2841

(57) ABSTRACT

Embodiments presented provide for a method for determining a hydrocarbon American Petroleum density in real-time. Aspects provide for using a formation testing apparatus to pump reservoir fluid and, based on downhole optical spectroscopy measurement as well as hydrocarbon density measurement determine the Americal Petroleum density.

### CROSS-REFERENCE TO RELATED APPLICATIONS

The present application claims priority to U.S. Provisional Application 63/759,669 dated Feb. 18, 2025, the entirety of which is incorporated by reference.

### FIELD OF THE DISCLOSURE

Aspects of the disclosure relate to optical spectroscopy. More specifically, aspects of the disclosure relate to use of optical spectroscopy to determine the specific gravity of fluids under the American Petroleum Institute (API) specific gravity scale.

### BACKGROUND

Hydrocarbons are integral to the functioning of modern society and industry. They serve as the primary source of energy for transportation, heating, and electricity generation, and are essential raw materials for countless products, including plastics, fertilizers, and chemicals. The widespread use of hydrocarbons has driven significant exploration and development activities, shaping industries and economies around the world. As time progresses, the prevalence of large, easily accessible hydrocarbon fields has diminished. Many of the world's most prolific fields have been depleted or are nearing the end of their productive lives, leaving fewer large reserves available for development.

The remaining hydrocarbon fields pose increasing challenges for exploration and production. These fields are often located at greater depths, in more remote or harsh environments, or contain fluids subjected to high temperatures and pressures. Developing such fields requires advanced technology and specialized expertise to address technical obstacles, such as drilling through deep formations, managing extreme reservoir conditions, and ensuring safe operations in isolated locations. Additionally, the complexity of these fields increases the risk of operational errors and complicates the assessment of their economic viability. As a result, field development has become more intricate and demanding for engineers and technical professionals.

Economic considerations play a pivotal role in determining which hydrocarbon fields are developed. The costs associated with exploration, drilling, production, and infrastructure development are substantial, especially for technically challenging fields. Generally, lower-cost fields are preferred, as they offer a higher likelihood of profitable operations and quicker returns on investment. Fields with higher development costs, due to depth, location, or reservoir complexity, are often deferred or abandoned unless market conditions justify their exploitation. This economic calculus is central to the decision-making process for energy companies and stakeholders.

The commodity price of oil is subject to frequent fluctuations, influenced by global supply and demand dynamics. Political events, economic cycles, technological advancements, and environmental regulations all contribute to changes in oil prices. When supply outpaces demand, prices fall, making high-cost field development less attractive. Conversely, when demand rises or supply is constrained, prices increase, incentivizing investment in more technically challenging and economically borderline fields. Over time, the cost of hydrocarbons has gradually increased, driven by resource scarcity and the need for more sophisticated extraction techniques. This trend underscores the importance of efficient and accurate field development methods.

Optical spectroscopy has emerged as a valuable tool in hydrocarbon field development, offering the ability to analyze the properties of fluids in situ and in real time. By examining the interaction between light and hydrocarbons, engineers can determine key characteristics such as composition, purity, and specific gravity. This technique allows for rapid and non-destructive analysis, supporting decision-making during drilling and production operations.

The benefits of optical spectroscopy include increased speed and efficiency in data collection, reduced reliance on laboratory testing, and improved safety through real-time monitoring. Field engineers can obtain critical information without waiting for sample transport and analysis, enabling more responsive and adaptive operations. The technology also facilitates the identification of fluid types and reservoir characteristics, helping to optimize production strategies and minimize risks.

Despite its advantages, optical spectroscopy is not without significant drawbacks. One major concern is the presence of errors in analyzing data related to fluid specific gravity. These errors can arise from instrument calibration, environmental conditions, or variations in fluid properties that are not adequately accounted for by the measurement system. Inaccurate readings may lead to incorrect assessments of reservoir quality and economic potential, potentially compromising field development decisions.

Another drawback is the challenge of converting spectroscopic data to standardized scales such as the API specific gravity scale. This conversion process is often complicated by differences in measurement techniques, the presence of impurities, and the lack of robust calibration standards. As a result, engineers may struggle to obtain reliable and consistent data, which can impact the accuracy of resource estimates and operational planning. The inability to seamlessly integrate spectroscopic measurements into established industry frameworks remains a critical limitation.

Conventional technologies used for hydrocarbon field development are frequently time-consuming and cumbersome for field engineers. Traditional methods involve extensive laboratory testing, sample preparation, and transportation, which can delay critical decision-making and increase operational costs. The complexity of these processes also contributes to the risk of human error and logistical challenges, further hindering field efficiency. In environments where rapid response is essential, these limitations can impede progress and affect overall project outcomes.

As the hydrocarbon industry confronts shrinking reserves and increasingly complex field conditions, the need for more economical and effective development methods is paramount. Optical spectroscopy offers promising capabilities for real-time fluid analysis, but current technologies face significant obstacles related to data accuracy, conversion to standard scales, and integration with conventional workflows. Addressing these drawbacks is essential to enhance the reliability and efficiency of field operations. The industry must continue to innovate and refine analytical techniques, striving for solutions that reduce costs, improve safety, and support sustainable resource development. By advancing beyond conventional technologies, engineers and professionals can better meet the challenges of modern hydrocarbon exploration and production.

There is a need to provide an apparatus and methods that are easier to operate than conventional apparatus and methods.

There is a further need to provide apparatus and methods that do not have the drawbacks discussed above.

There is a still further need to reduce economic costs associated with operations and apparatus described above with conventional tools.

### SUMMARY

So that the manner in which the above recited features of the present disclosure can be understood in detail, a more particular description of the disclosure, briefly summarized below, may be had by reference to embodiments, some of which are illustrated in the drawings. It is to be noted that the drawings illustrate only typical embodiments of this disclosure and are; therefore, not to be considered limiting of its scope, for the disclosure may admit to other equally effective embodiments without specific recitation. Accordingly, the following summary provides just a few aspects of the description and should not be used to limit the described embodiments to a single concept.

In one example embodiment, a method is disclosed. The method may comprise pumping a volume of reservoir fluid from a downhole environment to a fluid tester with a spectroscopy unit. The method may further comprise performing a hydrocarbon density measurement of the volume of reservoir fluid to produce density results. The method may further comprise determining a gas to oil ratio for the volume of reservoir fluid to produce gas to oil results. The method may further comprise determining an optical density for the volume of reservoir fluid to produce optical density results. The method may further comprise performing a composition weight fraction analysis for the reservoir fluid to produce composition weight fraction results. The method may further comprise normalizing the optical density results with the gas to oil results to produce first normalized results. The method may further comprise normalizing the optical density results with the density results to produce second normalized results. The method may further comprise normalizing the optical density results with the composition weight fraction results to produce third normalized results. The method may further comprise performing a summation of the first normalized results, the second normalized results, and the third normalized results to calculate a summed normalized fluid optical density property. The method may further comprise determining a hydrocarbon American Petroleum Institute density based upon the summed normalized fluid optical density property.

In another example embodiment, a method to calculate an American Petroleum Institute based specific gravity for a fluid, is disclosed. The method may comprise gathering data of at least one of a downhole fluid analyzer and a sampling station. The method may further comprise gathering pressure, volume, and temperature (PVT) laboratory data across the at least one sampling station while filtering measured American Petroleum Institute specific gravity values of the at least one station. The method may further comprise evaluating an optical density along with fluid properties of gas to oil ratio values, density values and composition values to achieve optical density results. The method may further comprise evaluating trends in the gas to oil ratio values obtained, the fluid composition obtained, and the density obtained with a measured American Petroleum Institute specific gravity value for multiple samples. The method may further comprise normalizing the optical density results with the gas to oil values from the gathered data to produce first normalized results. The method may further comprise normalizing the optical density results with the density values from the gathered data to produce second normalized results. The method may further comprise normalizing the optical density results with the composition weight fraction values to produce third normalized results. The method may further comprise performing a summation of the first normalized results, the second normalized results, and the third normalized results to calculate a normalized fluid optical density parameter. The method may further comprise plotting the normalized fluid optical density based American Petroleum Institute parameter with the measured American Petroleum Institute specific gravity. The method may further comprise fitting a trend line of the plotted normalized fluid optical density to establish a best fit equation. The method may further comprise calculating the American Petroleum Institute specific gravity for fluids at the at least one sampling station using the best fit equation.

In another example embodiment, an article of manufacture is disclosed. The article of manufacture may be configured to be read by a computing device, the article of manufacture configured with a list of instructions, the list of instructions configured to operate on the computing device, the list of instructions configured to perform a method comprising pumping a volume of reservoir fluid from a downhole environment to a fluid tester with a spectroscopy unit. The method described may also comprise performing a hydrocarbon density measurement of the volume of reservoir fluid to produce density results. The method described may also comprise determining a gas to oil ratio for the volume of reservoir fluid to produce gas to oil results. The method described may also comprise determining an optical density for the volume of reservoir fluid to produce optical density results. The method described may also comprise performing a composition weight fraction analysis for the reservoir fluid to produce composition weight fraction results. The method described may also comprise normalizing the optical density results with the gas to oil results to produce first normalized results. The method described may also comprise normalizing the optical density results with the density results to produce second normalized results. The method described may also comprise normalizing the optical density results with the composition weight fraction results to produce third normalized results. The method described may also comprise performing a summation of the first normalized results, the second normalized results, and the third normalized results to calculate a summed normalized fluid optical density property. The method described may also comprise determining a hydrocarbon American Petroleum Institute density based upon the summed normalized fluid optical density property.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the drawings. It is to be noted; however, that the appended drawings illustrate only typical embodiments of this disclosure and are; therefore, not be considered limiting of its scope, for the disclosure may admit to other equally effective embodiments.

FIG. 1 illustrates an example method for real-time API determination, according to one or more examples of the disclosure.

FIG. 2 illustrates a second example method for real-time API determination, according to one or more example of the disclosure.

To facilitate understanding, identical reference numerals have been used, where possible, to designate identical elements that are common to the figures (“FIGS”). It is contemplated that elements disclosed in one embodiment may be beneficially utilized on other embodiments without specific recitation.

### DETAILED DESCRIPTION

In the following, reference is made to embodiments of the disclosure. It should be understood; however, that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments, and advantages are merely illustrative and are not considered elements or limitations of the claims except where explicitly recited in a claim. Likewise, reference to “the disclosure” shall not be construed as a generalization of inventive subject matter disclosed herein and should not be considered to be an element or limitation of the claims except where explicitly recited in a claim.

Although the terms first, second, third, etc., may be used herein to describe various elements, components, regions, layers and/or sections, these elements, components, regions, layers, and/or sections should not be limited by these terms. These terms may be only used to distinguish one element, components, region, layer, or section from another region, layer, or section. Terms such as “first”, “second”, and other numerical terms, when used herein, do not imply a sequence or order unless clearly indicated by the context. Thus, a first element, component, region, layer, or section discussed herein could be termed a second element, component, region, layer, or section without departing from the teachings of the example embodiments.

When an element or layer is referred to as being “on”, “engaged to”, “connected to”, or “coupled to” another element or layer, it may be directly on, engaged, connected, coupled to the other element or layer, or interleaving elements or layers may be present. In contrast, when an element is referred to as being “directly on”, “directly engaged to”, “directly connected to”, or “directly coupled to” another element or layer, there may be no interleaving elements or layers present. Other words used to describe the relationship between elements should be interpreted in a like fashion. As used herein, the term “and/or” includes any and all combinations of one or more of the associated listed terms.

Some embodiments will now be described with reference to the figures. Like elements in the various figures will be referenced with like numbers for consistency. In the following description, numerous details are set forth to provide an understanding of various embodiments and/or features. It will be understood; however, by those skilled in the art, that some embodiments may be practiced without many of these details, and that numerous variations or modifications from the described embodiments are possible. As used herein, the terms “above” and “below”, “up” and “down”, “upper” and “lower”, “upwardly” and “downwardly”, and other like terms indicating relative positions above or below a given point are used in this description to more clearly describe certain embodiments.

Example embodiments of the disclosure are directed towards a method for real time downhole API gravity scale determinations. Embodiments of the present disclosure may be further directed toward a gravity scale for measuring of relative density of various petroleum liquids. Such liquids may include, for example, pure liquids and liquid gas combinations. These determinations may be performed in real-time at the wellbore or wellsite by field engineers.

Aspects of the disclosure may be performed by a combination of field components, such as pumps and downhole optical measurement devices. Such optical measurement devices may be, for example, placed inside a formation testing apparatus. Non-limiting example embodiments include downhole spectroscopy units.

Optical spectroscopy units operate by analyzing the interaction between light and matter, typically within the visible, ultraviolet, or infrared spectrum. These units emit light through a sample and detect the resulting absorption, transmission, or reflection to determine the sample's unique spectral signature. This process allows for precise identification and measurement of molecular components based on the wavelengths and intensities recorded.

In downhole applications, optical spectroscopy units are integrated into field equipment to provide real-time analysis of petroleum liquids and gas combinations. By capturing spectral data directly at the wellsite, engineers can quickly assess the relative density and composition of fluids without the need for extensive laboratory testing. This immediate feedback streamlines decision-making during formation testing and enhances the efficiency of wellbore operations. While helpful, optical spectroscopy does not allow for direct hydrocarbon API density providing a significant shortfall for existing technology.

The gas to oil ratio (GOR) is a critical parameter in petroleum engineering that quantifies the amount of gas produced relative to the amount of oil from a reservoir. It is typically expressed in standard cubic feet of gas per barrel of oil (SCF/bbl). GOR provides insight into the characteristics of the reservoir fluid and is essential for evaluating reservoir performance, designing production strategies, and forecasting reserves. High GOR values may indicate the presence of volatile oil or gas condensate reservoirs, while lower values are generally associated with black oil systems.

In field conditions, GOR is measured by collecting wellsite samples of reservoir fluids and separating the gas from the liquid phase using a test separator. The produced fluids are directed into the separator, where the gas and oil are isolated and measured independently. Flow meters and calibrated tanks are used to quantify the volumes of gas and oil produced over a specific time interval. The ratio is then calculated by dividing the measured gas volume by the corresponding oil volume under standard temperature and pressure conditions. This real-time determination of GOR enables field engineers to make informed decisions regarding production optimization and reservoir management.

Presently disclosed embodiments include a method for determining hydrocarbon API density in real time, including using a formation tester to pump reservoir fluid, based on downhole spectroscopy measurement and hydrocarbon density measurements, measuring, while the reservoir fluid is being pumped, properties including gas-to oil ratio (GOR), density and composition weight fraction (C1, C2, C3, C4, C5, C6+, and CO₂) across a clean (representative slug). The optical density data is normalized with the GOR, the fluid density, and with individual fluid compositions (C1, C2, C3, C4, C5, C6+ and CO₂). Next, the normalized optical densities (with reference to GOR, density and composition) are summated to calculate normalized fluid OD property to determine API density. As will be understood, the composition weight fraction is defined as the amount of weight of each specific component to the total mass of the structure.

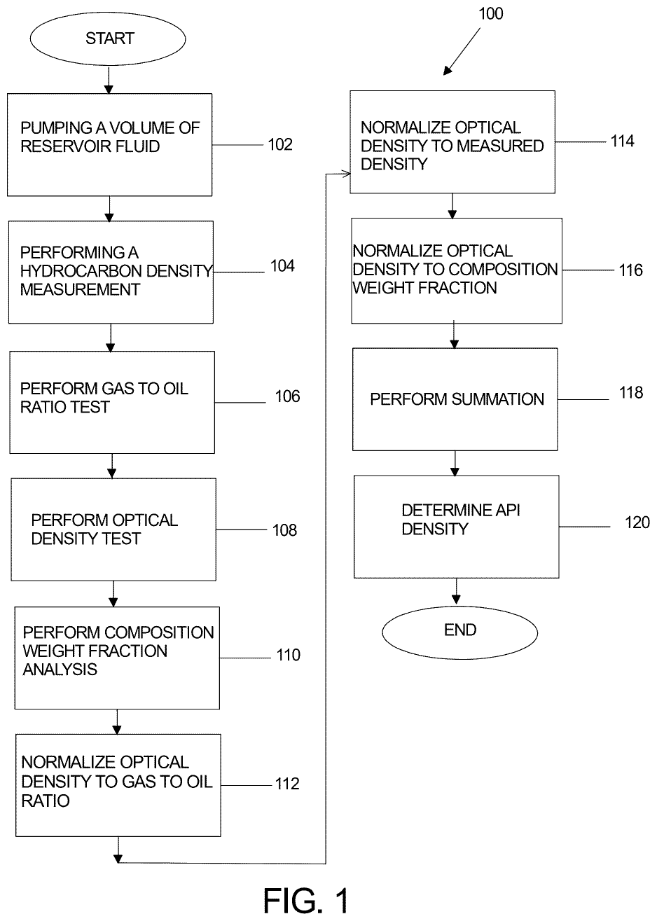

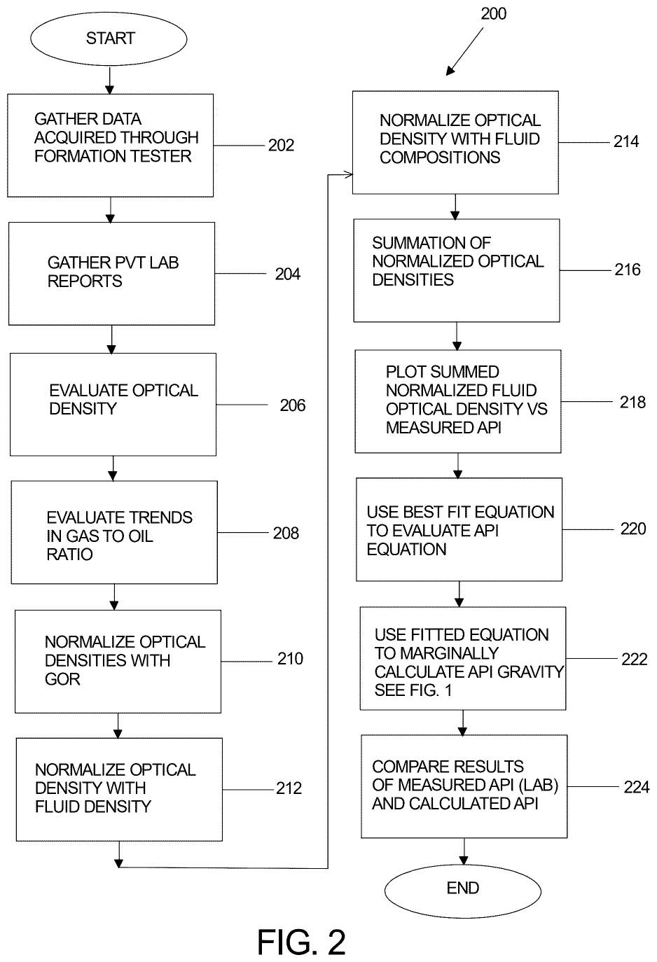

Referring to FIG. 1, a method 100 is illustrated for determining hydrocarbon API density in real time. As will be understood, the method in FIG. 1 may be performed after execution of method steps provided in FIG. 2. As will be further understood, this method may be accomplished at the field site instead of inside a laboratory as done with conventional methods. At 102, the method provides for pumping a volume of reservoir fluid from a downhole environment to a fluid tester with a spectroscopy unit. At 104, the method provides for performing a hydrocarbon density measurement of the volume of reservoir fluid to produce density results. At 106, the method provides for determining an gas to oil ratio for the volume of reservoir fluid to produce gas to oil results. At 108, the method provides for determining an optical density for the volume of reservoir fluid to produce optical density results. At 110, the method provides for performing a composition weight fraction analysis for the reservoir fluid to produce composition weight fraction results. At 112, the method provides for normalizing the optical density results with the gas to oil results to produce first normalized results. At 114, the method provides for normalizing the optical density results with the density results to produce second normalized results. At 116, the method provides for normalizing the optical density results with the composition weight fraction results to produce third normalized results. At 118, the method provides for performing a summation of the first normalized results, the second normalized results, and the third normalized results to calculate a summed normalized fluid optical density property. At 120, the method provides for determining a hydrocarbon American Petroleum Institute density based upon the summed normalized fluid optical density property.

In one or more embodiments, the determined API is gradated in degrees on a hydrometer instrument and is configured so that most values fall between 10 degrees and 70 degrees API gravity. The arbitrary formula to obtain such values may be: API gravity—141.5/SG at 60 degrees F—131.5 where SD is the specific gravity of the fluid.

As will be understood, a modular downhole fluid analysis may be used to conduct testing. Hydrocarbon composition (C1-C6+) may be conducted by a reservoir fluid composition sensor. In embodiments, the fluid composition measurements include both heavy and light ends. Algorithms used characterize the C6+ group on a basis of wax and branched-alkane content. Corrections for spectrum variation may be performed in some embodiments.

In embodiments, ethane (C2) may be determined separately from other carbon groups. In some embodiments, the sensors used may use dual spectrometers for spectrums of visible light through near infrared light. In further embodiments, Beer-Lambert law may be used to indicate the optical absorption of a component that is proportional to its concentration.

In further embodiments, GOR sensors may be used in the downhole testing platform. In embodiments, a filter array is used as well as a grating with an optical spectrometer. Such filters and gratings may be optimized for detection and analysis of hydrocarbons and carbon dioxide components in crude oil and/or other formation fluids. As will be understood, GOR is a fluid purity indicator. This value represents the level of contamination in a reservoir fluid that has been extracted from the formation.

In still further embodiments, carbon dioxide content may be determined through a reservoir fluid carbon dioxide sensor. As will be understood, determination of reliable quantification of carbon dioxide values from reservoir fluid samples may be difficult to obtain. Carbon dioxide readily reacts with water, whether from a mud filtrate contamination or through formation water contact. In embodiments, using a dedicated filter array and grating optical spectrometry system, upper and lower accuracy tolerances may be provided for use in subsequent evaluation.

Referring to FIG. 2, a second method is illustrated. As will be understood, the method provided in FIG. 2, may be accomplished separately and/or before the method performed in FIG. 1. The method 200 relates to calculating an American Petroleum Institute based specific gravity for a fluid. The method provides, at 202, gathering data of at least one of a downhole fluid analyzer and a sampling station. The method may further comprise, at 204, gathering pressure, volume, and temperature laboratory data across the at least one sampling station while filtering measured American Petroleum Institute specific gravity values of the at least one station. The method may further comprise, at 206, evaluating an optical density along with fluid properties of gas to oil ratio values, density values and composition values to achieve optical density results. The method may further comprise, at 208, evaluating trends in the gas to oil ratio values obtained, the fluid composition obtained and the density obtained with a measured American Petroleum Institute specific gravity value for multiple samples. The method may further comprise, at 210, normalizing the optical density results with the gas to oil values from the gathered data to produce first normalized results. The method may further comprise, at 212, normalizing the optical density results with the density values from the gathered data to produce second normalized results. The method may further comprise, at 214, normalizing the optical density results with the composition weight fraction values to produce third normalized results. The method may further comprise, at 216, performing a summation of the first normalized results, the second normalized results and the third normalized results to calculate a summed normalized fluid optical density parameter. The method may further comprise, at 218, plotting the normalized fluid optical density based American Petroleum Institute parameter with the measured American Petroleum Institute specific gravity. The method may further comprise, at 220, fitting a trend line of the plotted normalized fluid optical density to establish a best fit equation. The method may further comprise, at 222, calculating the American Petroleum Institute specific gravity for fluids at the at least one sampling station using the best fit equation. The method may also comprise comparing results of the measured specific gravity (within a laboratory) to the calculated specific gravity at 224.

In some embodiments, methods described may be stored in a non-volatile memory. In some embodiments, the non-volatile memory may be defined as an article of manufacture. In embodiments, the non-volatile memory is configured such that the methods may contain a list of instructions that may be read by a computing device and the list of instructions performed. The list of instructions may perform calculations, illustrate graphic results on a visual device, such as a monitor, print results or store data for further use, as non-limiting embodiments. The list of instructions may be executable in their own programming or may be executed using other programming. The list of instructions may be stored in various configurations, such as a compact disk, a floppy disk, a solid-state drive, a computer hard drive, a server, a web-oriented storage device, and a cloud-computing device or system. Embodiments of methods described may control other systems, such as machines, to perform specified functions. Operational control may be performed through additional programming and/or operation of other computing or control devices. Embodiments described may be implemented using wireless technologies to allow for computing and execution of the list of instructions from various locations. Computing may occur, for example, in various platforms, including a personal computer, a laptop computer, a computer server, a cloud-based computer, a mainframe computer, a cellular telephone and a cellular connected device.

Embodiments of the methods described may use other programming technologies to help implement the methods described. In some embodiments, machine learning programming may be used to evaluate data and provide results. In some embodiments, training datasets may be used to allow for convergence of needed results and thus using pretrained machine learning programming is considered within the scope of the disclosure. In other instances, artificial intelligence programming systems may be implemented as part of the disclosure or may be incorporated within the methods described. Such artificial intelligence systems may be used in various capacities, including results generation, error detection, problem definition, and problem convergence methods. Graphical representation of results obtained by artificial intelligence systems is also considered within the scope of the disclosure.

In embodiments using machine learning and/or artificial intelligence, programming may be altered by the programming based upon instructions provided. As such, in one non-limiting embodiment, different nodal layers of evaluation may be provided for analysis. The different nodal layers provided may incorporate modification techniques to allow for accurate reading and evaluation of large datasets. The large datasets may be designated training datasets or may be actual data that is desired to be evaluated. Coefficients used for corresponding different nodal layers may be developed within the methods described or may be pre-set according to training. Such coefficients may be altered by the computer programming itself or may be designated by a computer user. As a non-limiting embodiment, if possible results from analysis disclose too many potential outcomes or results, a computer operator may be asked or may alter the analysis protocol to achieve more focused results.

In embodiments, computer code may be any programming code that lists instructions to be followed. Programming codes may include instructions provided by a computer programmer with or without assistance by computers. Programming may occur through use of a library of programs or subroutines to section programming tasks. Programming may be accomplished to run on different operating systems or may be included with internal executable files for stand-alone computer instructions.

Example embodiments of the disclosure are described next. The example embodiments should not be considered limiting. In one example embodiment, a method is disclosed. The method may comprise pumping a volume of reservoir fluid from a downhole environment to a fluid tester with a spectroscopy unit. The method may further comprise performing a hydrocarbon density measurement of the volume of reservoir fluid to produce density results. The method may further comprise determining a gas to oil ratio for the volume of reservoir fluid to produce gas to oil results. The method may further comprise determining an optical density for the volume of reservoir fluid to produce optical density results. The method may further comprise performing a composition weight fraction analysis for the reservoir fluid to produce composition weight fraction results. The method may further comprise normalizing the optical density results with the gas to oil results to produce first normalized results. The method may further comprise normalizing the optical density results with the density results to produce second normalized results. The method may further comprise normalizing the optical density results with the composition weight fraction results to produce third normalized results. The method may further comprise performing a summation of the first normalized results, the second normalized results and the third normalized results to calculate a summed normalized fluid optical density property. The method may further comprise determining a hydrocarbon American Petroleum Institute density based upon the normalized fluid optical density property.

In another example embodiment, the method may further comprise placing the formation tester into a wellbore prior to pumping the volume of reservoir fluid from the downhole environment.

In another example embodiment, the method may be performed wherein the placing of the formation tester is performed on a wireline.

In another example embodiment, the method may be performed wherein the determining the hydrocarbon American Petroleum Institute density is calculated through a formula:

$$API gravity = 141.5 / SG where SG is a specific gravity of the fluid .$$

In another example embodiment, the method may be performed wherein the API gravity the value 141.5/SG is at 60 degrees F.

In another example embodiment, the method may be performed wherein the performing the composition weight fraction analysis includes values for C1, C2, C3, C4, C5 and C6+.

In another example embodiment, the method may be performed wherein the performing the composition weight fraction analysis includes carbon dioxide.

In another example embodiment, the method may further comprise at least one of displaying and storing the hydrocarbon American Petroleum Institute density calculated.

In another example embodiment, the method may be performed wherein the determining the hydrocarbon American Petroleum Institute density based upon the normalized fluid optical density property occurs at a wellsite.

In another example embodiment, a method to calculate an American Petroleum Institute based specific gravity for a fluid, is disclosed. The method may comprise gathering data of at least one of a downhole fluid analyzer and a sampling station. The method may further comprise gathering pressure, volume and temperature laboratory data across the at least one sampling station while filtering measured American Petroleum Institute specific gravity values of the at least one station. The method may further comprise evaluating an optical density along with fluid properties of gas to oil ratio values, density values and composition values to achieve optical density results. The method may further comprise evaluating trends in the gas to oil ratio values obtained, the fluid composition obtained and the density obtained with a measured American Petroleum Institute specific gravity value for multiple samples. The method may further comprise normalizing the optical density results with the gas to oil values from the gathered data to produce first normalized results. The method may further comprise normalizing the optical density results with the density values from the gathered data to produce second normalized results. The method may further comprise normalizing the optical density results with the composition weight fraction values to produce third normalized results. The method may further comprise performing a summation of the first normalized results, the second normalized results and the third normalized results to calculate a summed normalized fluid optical density parameter. The method may further comprise plotting the summed normalized fluid optical density versus the measured American Petroleum Institute specific gravity. The method may further comprise fitting a trend line of the plotted summed normalized fluid optical density vs API to establish a best fit equation. The method may further comprise calculating the American Petroleum Institute specific gravity for fluids at the at least one sampling station using the best fit equation.

In another example embodiment, the method may be performed wherein the pressure, volume, and temperature data gathered is performed in the near infra-red conditions.

In another example embodiment, the method may be performed wherein the pressure, volume, and temperature data gathered is performed in a visible light.

In another example embodiment, the method may be performed wherein the measured American Petroleum Institute sample is in a laboratory.

In another example embodiment, the method may further comprise comparing results of the measured American Petroleum Institute specific gravity and the calculated American Petroleum Institute specific gravity.

In another example embodiment, the method may be performed wherein the gathering of the data of the at least one of the downhole fluid analyzer and the sampling station is through use of a formation tester.

In another example embodiment, the method may be performed wherein the at least one station is multiple stations.

In another example embodiment, the method may be performed wherein the filtering includes obtaining an American Petroleum Institute specific gravity value for each station.

In another example embodiment, the method further comprises at least one of displaying and storing the calculated American Petroleum Institute specific gravity values calculated.

In another example embodiment, an article of manufacture is disclosed. The article of manufacture may be configured to be read by a computing device, the article of manufacture configured with a list of instructions, the list of instructions configured to operate on the computing device, the list of instructions configured to perform a method comprising pumping a volume of reservoir fluid from a downhole environment to a fluid tester with a spectroscopy unit. The method described may also comprise performing a hydrocarbon density measurement of the volume of reservoir fluid to produce density results. The method described may also comprise determining an gas to oil ratio for the volume of reservoir fluid to produce gas to oil results. The method described may also comprise determining an optical density for the volume of reservoir fluid to produce optical density results. The method described may also comprise performing a composition weight fraction analysis for the reservoir fluid to produce composition weight fraction results. The method described may also comprise normalizing the optical density results with the gas to oil results to produce first normalized results. The method described may also comprise normalizing the optical density results with the density results to produce second normalized results. The method described may also comprise normalizing the optical density results with the composition weight fraction results to produce third normalized results. The method described may also comprise performing a summation of the first normalized results, the second normalized results and the third normalized results to calculate a summed normalized fluid optical density property. The method described may also comprise determining a hydrocarbon American Petroleum Institute density based upon the summed normalized fluid optical density property.

In another example embodiment, the article of manufacture may be configured in a form of a compact disk, a solid-state drive, a computer hard drive, a universal serial bus device and a non-volatile memory arrangement.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may be varied in many ways. Such variations are not to be regarded as a departure from the disclosure, and all such modifications are intended to be included within the scope of the disclosure.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

What is claimed is:

1. A method, comprising:
pumping a volume of reservoir fluid from a downhole environment to a fluid tester with a spectroscopy unit;
performing a hydrocarbon density measurement of the volume of reservoir fluid to produce density results;
determine a gas to oil ratio for the volume of reservoir fluid to produce gas to oil results;
determine an optical density for the volume of reservoir fluid to produce optical density results;
performing a composition weight fraction analysis for the reservoir fluid to produce composition weight fraction results;
normalizing the optical density results with the gas to oil results to produce first normalized results;
normalizing the optical density results with the density results to produce second normalized results;
normalizing the optical density results with the composition weight fraction results to produce third normalized results;
performing a summation of the first normalized results, the second normalized results, and the third normalized results to calculate a summed normalized fluid optical density property; and
determining a hydrocarbon American Petroleum Institute density based upon the summed normalized fluid optical density property.

2. The method according to claim 1, further comprising:
placing the formation tester into a wellbore prior to pumping the volume of reservoir fluid from the downhole environment.

3. The method according to claim 2, wherein the placing of the formation tester is performed on a wireline.

4. The method according to claim 1, wherein the determining the hydrocarbon American Petroleum Institute density is calculated through a formula:
$$API gravity = 141.5 / SG where SG is a specific gravity of the fluid .$$

5. The method according to claim 4, wherein the API gravity the value 141.5/SG is at 60 degrees F.

6. The method according to claim 1, wherein the performing the composition weight fraction analysis includes values for C1, C2, C3, C4, C5 and C6+.

7. The method according to claim 6, wherein the performing the composition weight fraction analysis includes carbon dioxide.

8. The method according to claim 1, further comprising:
at least one of displaying and storing the hydrocarbon American Petroleum Institute density calculated.

9. The method according to claim 1, wherein the determining the hydrocarbon American Petroleum Institute density based upon the normalized fluid optical density property occurs at a wellsite.

10. A method to calculate an American Petroleum Institute based specific gravity for a fluid, comprising:
gathering data of at least one of a downhole fluid analyzer and a sampling station;
gathering pressure, volume and temperature laboratory data across the at least one sampling station while filtering measured American Petroleum Institute specific gravity values of the at least one station;
evaluating an optical density along with fluid properties of gas to oil ratio values, density values and composition values to achieve optical density results;
evaluating trends in the gas to oil ratio values obtained, the fluid composition obtained and the density obtained with a measured American Petroleum Institute specific gravity value for multiple samples;
normalizing the optical density results with the gas to oil values from the gathered data to produce first normalized results;
normalizing the optical density results with the density values from the gathered data to produce second normalized results; and
normalizing the optical density results with the composition weight fraction values to produce third normalized results;
performing a summation of the first normalized results, the second normalized results and the third normalized results to calculate a summed normalized fluid optical density;
plotting the summed normalized fluid optical density versus the measured American Petroleum Institute specific gravity;
fitting a trend line of the plotted normalized fluid optical density vs API to establish a best fit equation; and
calculating the American Petroleum Institute specific gravity for fluids at the at least one sampling station using the best fit equation.

11. The method according to claim 10, wherein the pressure, volume, and temperature data gathered is performed in the near infra-red conditions.

12. The method according to claim 10, wherein the pressure, volume, and temperature data gathered is performed in a visible light.

13. The method according to claim 10, wherein the measured American Petroleum Institute sample is in a laboratory.

14. The method according to claim 10, further comprising comparing results of the measured American Petroleum Institute specific gravity and the calculated American Petroleum Institute specific gravity.

15. The method according to claim 10, where the gathering of the data of the at least one of the downhole fluid analyzer and the sampling station is through use of a formation tester.

16. The method according to claim 10, wherein the at least one station is multiple stations.

17. The method according to claim 10, wherein the filtering includes obtaining an American Petroleum Institute specific gravity value for each station.

18. The method according to claim 10, further comprising at least one of displaying and storing the calculated American Petroleum Institute specific gravity values calculated.

19. An article of manufacture configured to be read by a computing device, the article of manufacture configured with a list of instructions, the list of instructions configured to operate on the computing device, the list of instructions configured to perform a method comprising:
pumping a volume of reservoir fluid from a downhole environment to a fluid tester with a spectroscopy unit;
performing a hydrocarbon density measurement of the volume of reservoir fluid to produce density results;
determining a gas to oil ratio for the volume of reservoir fluid to produce gas to oil results;
determining an optical density for the volume of reservoir fluid to produce optical density results;
performing a composition weight fraction analysis for the reservoir fluid to produce composition weight fraction results;
normalizing the optical density results with the gas to oil results to produce first normalized results;
normalizing the optical density results with the density results to produce second normalized results;
normalizing the optical density results with the composition weight fraction results to produce third normalized results;
performing a summation of the first normalized results, the second normalized results and the third normalized results to calculate a summed normalized fluid optical density property; and
determining a hydrocarbon American Petroleum Institute density based upon the summed normalized fluid optical density property.

20. The article of manufacture, according to claim wherein the article of manufacture is in a form of a compact disk, a solid-state drive, a computer hard drive, a universal serial bus device and a non-volatile memory arrangement.
